(19) United States
(12) Patent Application Publication
(10) Pub. No.: US 2018/0286521 A1
(43) Pub. Date: Oct. 4, 2018

(54) PERI-OPERATIVE REMOTE CARE MONITORING SYSTEM

(71) Applicant: City of Hope, Duarte, CA (US)

(72) Inventor: Yuman Fong, La Canada, CA (US)

(21) Appl. No.: 15/942,953

(22) Filed: Apr. 2, 2018

Related U.S. Application Data
(60) Provisional application No. 62/480,922, filed on Apr. 3, 2017.

(51) Int. Cl. G16H 80/00; G16H 20/40

(52) U.S. Cl. CPC G16H 80/00; G16H 20/40

(57) ABSTRACT

A system is configured to assist a caregiver in remotely monitoring a patient that has undergone a medical procedure such as surgery. The system is used pursuant to an enhanced recovery after surgery (ERAS) protocol and can be used to assemble and record data for effective triage of patients. The system can be used to optimize or otherwise improve post-operative care of the patient. The system can assist in monitoring or preventing complications of the patient after the patient leaves a care facility, such as a hospital.

### CROSS REFERENCE TO RELATED APPLICATION

This application claims priority to U.S. patent application Ser. No. 62/480,922, filed on Apr. 3, 2017 and entitled “Peri-Operative Remote Care Monitoring System”. Priority to the aforementioned filing date is claimed and the provisional patent application is incorporated herein by reference in its entirety.

### BACKGROUND

Due to changes in the current healthcare system as well as advances in surgical procedures, patients are being discharged earlier and earlier from the hospital after surgery relative to discharge standards in prior times. As a result, there is a possibility that patients may not receive proper care after surgery. This can often lead to readmission of a patient into a care facility or hospital due to complications resulting from inadequate monitoring of the patient after the initial discharge of the patient. This can be very inconvenient for the patient and can also be costly from a monetary standpoint for both the hospital and the patient.

There is a need for innovative care systems that result in safer early discharge, address the needs of pre-operative, post-operative, and post-discharge care, and achieve improved care of a patient in the post-operative setting.

### SUMMARY

A system is configured to assist in remote care and monitoring of a peri-operative patient. The system includes a wearable device that remotely monitors or senses data related to health parameters and that also collects quality-of-life parameters of the patient. The system is configured to securely transmit such data to a remote facility via a data network for storage and archiving data. The system includes a program or software application that takes the data obtained and displays trending activities for clinical use. This includes triggers for emergent and urgent interventions by personnel in the monitoring center as well as clinical pathways for care. physician or other caregiver can monitor and evaluate the data to effectuate proper care of the patient pursuant to an Enhanced Recovery After Surgery (ERAS) pathway.

The elements of the system may vary and can include, for example, a mobile device (such as a smartphone or a wireless wearable device), a mobile application on the device, a secure data transmission and storage mechanism, and a program for efficient display of data for monitoring and clinical use. In an embodiment, the system includes a mobile application that effectuates real-time symptom monitoring of the patient. In addition, a remote platform data center data is configured to analyze the data such as for trends that indicate that care of the patient should be modified. The remote platform data center is configured to issue an alert to the patient or caregiver to indicate that an action needs to be taken with respect to the patient to effectuate proper care.

In an example method, the system is used in connection with a patient or patients having major abdominal cancer surgery. An example patient wears a wristband pedometer and also completes a patient-reported outcome survey, such as via an online or network interface. The survey includes patient-reported data related to symptoms and quality of life. The patient can complete and report the surveys prior to surgery (such as 3-7 days before surgery), during hospitalization related to the surgery, and after surgery (such as about 2 weeks after discharge from a hospital.) The system can collect data via electronic medical records and calculate complications, if any, using the Clavien-Dindo classification. Pursuant to this example method, the monitoring facility and/or the physician can communicate with the patient (such as via email or text message) such as to generate reminders or notices to the patient when the metric indicates an issue with respect to measured symptoms and/or quality of life.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic representation of a peri-operative remote care monitoring system.

FIG. 2 shows a schematic representation of a wearable device that is part of the monitoring system of FIG. 1.

### DETAILED DESCRIPTION

Disclosed is a system that is configured to assist a caregiver, such as a physician, in remotely monitoring a patient that has undergone a medical procedure such as surgery. In an embodiment, the system is used pursuant to an enhanced recovery after surgery (ERAS) protocol and can be used to assemble and record data for effective triage of patients. The system can be used to optimize or otherwise improve post-operative care of the patient. The system can assist in monitoring or preventing complications of the patient after the patient leaves a care facility, such as a hospital. For example, the system can monitor the patient at home after surgery and can assist in preventing or alleviating complications after surgery to thereby reduce the likelihood of readmission into the care facility due to such complications.

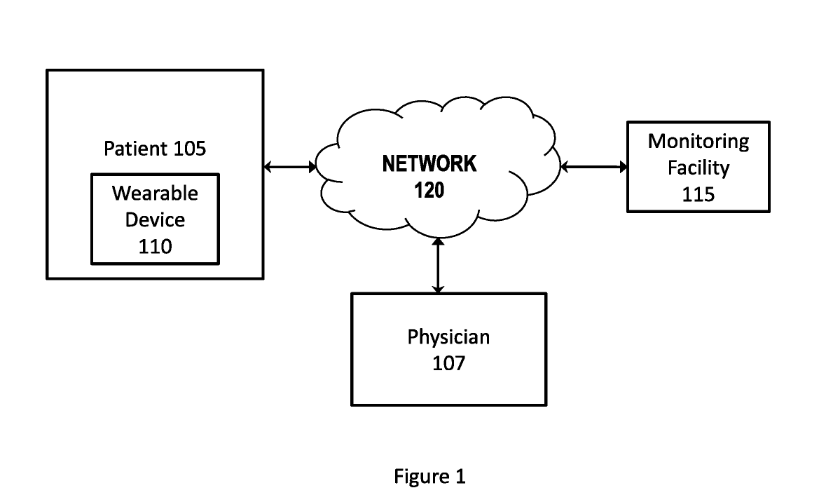

FIG. 1 shows a schematic representation of a system that is configured to assist a patient 105 and/or a caregiver, such as a physician 107, in monitoring care of the patient 105 in a peri-operative setting, such as after a surgical procedure of the patient. The system includes a wearable device 110 (also referred to as a monitor device) that is worn or otherwise communicatively coupled to or by the patient 105 and that assists in automatically and remotely sensing or monitoring one or more aspects of the patient, as described more fully below. The system further includes a monitoring facility 115 that serves as a data center and includes one or more computing systems for collecting and/or analyzing the data. A communication network 120 is communicatively coupled to the wearable device 110 and to the monitoring facility 115. The wearable device 110 can transmit data to the monitoring facility 115 (such as via the network 120 or via a direct connection to the monitoring facility 115) wherein the data is related to a physical parameter of the patient 110. The physician 107 can access the data via the network 120 or via a direct connection to the monitoring facility 115 and can act on the data to effectuate proper care of the patient based on analysis of the data.

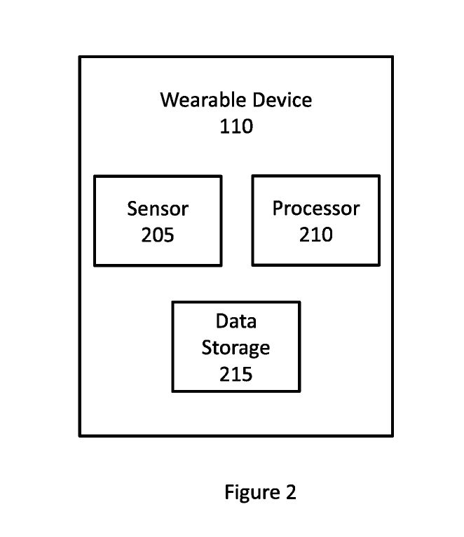

FIG. 2 shows a schematic representation of the wearable device 110, which is a device that can be worn by the patient 105 such as on the patient's wrist or other part of the patient's body. The device includes at least one sensor 205 that is adapted to sense a physical parameter or health parameter of the patient. The physical parameter or health parameter can vary and can include, for example, blood pressure and heart rate. In an embodiment, the wearable device 110 is a pedometer and it is adapted to sense walking activity of a patient, such as quantity of walking strides that a patient takes per unit time.

In an embodiment, the health parameter relates to at least one of the following: a heart rate of the post-operative patient, skin or ambient temperature of the post-operative patient, body position of the post-operative patient, ECG of the post-operative patient, physical activity of the post-operative patient, daily walking steps of the post-operative patient, pulse oximetry of the post-operative patient, blood pressure of the post-operative patient, sleep activity of the post-operative patient, and blood glucose of the post-operative patient.

The wearable device 110 also includes a computer processor 210 and/or data storage 215 that permits the wearable device 110 to store data and, in conjunction with the computer processor 210, to process computer commands such as in the form of software. In an embodiment, at least one mobile software application is loaded on the wearable device 110. The mobile application is configured to effectuate any of the features described herein, such as the recording and transfer of data via the network 120.

The patient 105 can also manually enter other data into the application via the wearable device 110 or via a separate device that can be coupled to the wearable device. For example a separate device can be a mobile phone, digital assistant, or a computer. In an embodiment, the patient can enter data related to or indicative of a quality-of-life of the patient in the postoperative setting. In an embodiment, such quality-of-life data relates to at least one of the following: mobility of the post-operative patient, self-care of the post-operative patient, usual activities of the post-operative patient, pain and discomfort of the post-operative patient, and anxiety or depression of the post-operative patient.

The mobile application is configured to monitor the post-operative patient 105 and include software that is configured to receive the health parameter data from the wearable device 110 device attached to the post-operative patient. The health parameter data is automatically generated by the wearable device 110. The mobile application can also receive the quality of life data from the post-operative patient and send the health parameter data and the quality of life data to the data center 115 via the network 120 for analysis related to post-operative care of the post-operative patient.

The monitoring facility 115 receives the health parameter data from the monitor device attached to the post-operative patient and also receives quality of life data from the post-operative patient, the quality of life data being generated by the post-operative patient. The monitoring facility can include a computing capability that analyzes the heath parameter data and the quality of life data to generate a metric. This metric can be compared to a predetermined alert threshold. An alert can be issued upon the metric exceeding the predetermined alert threshold. Alternatively or in conjunction with the monitoring facility automatically analyzing the data, the physician 107 can also access the data and perform a manual analysis of the data including an analysis of any trends in the data.

In an example embodiment, the monitoring facility or some other aspect of the system uses an artificial intelligence (AI) hardware or software to analyze the data and/or generate the metric. The AI component can also issue a communication to the patient based on the analysis and/or the metric.

To the extent that the monitoring facility or the physician issues an alert, the alert can prompt a healthcare professional to contact the post-operative patient. The alert may also prompt a healthcare professional to perform an assessment of at least one of the quality of life data and the heath parameter data. In the situation where the health parameter data comprises data related to a quantity of physical waking of the post-operative patient, the alert can be issued if the quantity of physical waking is below a walking threshold.

As mentioned, the monitoring facility 115 or the physician 107 can analyze and tabulate trends in the quality of life data and the health parameter data. In addition, it can be verified that the post-operative patient recently underwent a surgical procedure as a condition of the patient 105 having access to the wearable device 110 and/or the application.

In an embodiment, the data is analyzed pursuant to MD Anderson Symptom Inventory (MDASI). In an embodiment, patient complications related to the data is analyzed pursuant to the Clavien-Dindo classification.

In an example method, the system is used in connection with a patient or patients having major abdominal cancer surgery. An example patient wears a wristband pedometer and also completes a patient-reported outcome survey, such as via an online or network interface. The survey includes patient-reported data related to symptoms and quality of life. The patient can complete and report the surveys prior to surgery (such as 3-7 days before surgery), during hospitalization related to the surgery, and after surgery (such as about 2 weeks after discharge from a hospital.) The system can collect data via electronic medical records and calculate complications, if any, using the Clavien-Dindo classification. Pursuant to this example method, the monitoring facility and/or the physician can communicate with the patient (such as via email or text message) such as to generate reminders or notices to the patient when the metric indicates an issue with respect to measured symptoms and/or quality of life.

One or more aspects or features of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device (e.g., mouse, touch screen, etc.), and at least one output device.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid state memory or a magnetic hard drive or any equivalent storage medium. The machine-readable medium can alternatively or additionally store such machine instructions in a transient manner, such as for example as would a processor cache or other random access memory associated with one or more physical processor cores.

With certain aspects, to provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), the Internet, WiFI (IEEE 802.11 standards), NFC, BLUETOOTH, ZIGBEE, and the like.

The computing system may include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A method of monitoring care of a post-operative patient, comprising:
receiving health parameter data from a monitor device attached to the post-operative patient, the health parameter data being automatically generated by the monitor device and being indicative of a physical parameter of the post-operative patient;
receiving quality of life data from the post-operative patient, the quality of life data being generated by the post-operative patient;
analyzing the heath parameter data and the quality of life data to generate a metric;
comparing the metric to a predetermined alert threshold;
causing an alert to be issued upon the metric exceeding the predetermined alert threshold.

2. A method as in claim 1, wherein the alert prompts a healthcare professional to contact the post-operative patient.

3. A method as in claim 1, wherein the alert prompts a healthcare professional to perform an assessment of at least one of the quality of life data and the heath parameter data.

4. A method as in claim 1, wherein the health parameter data comprises at least one of a heart rate of the post-operative patient, skin or ambient temperature of the post-operative patient, body position of the post-operative patient, ECG of the post-operative patient, physical activity of the post-operative patient, daily walking steps of the post-operative patient, pulse oximetry of the post-operative patient, blood pressure of the post-operative patient, sleep activity of the post-operative patient, and blood glucose of the post-operative patient.

5. A method as in claim 1, wherein the quality of life data comprises at least one of data related to mobility of the post-operative patient, self-care of the post-operative patient, usual activities of the post-operative patient, pain and discomfort of the post-operative patient, and anxiety or depression of the post-operative patient.

6. A method as in claim 1, wherein the health parameter data comprises data related to a quantity of physical waking of the post-operative patient, and wherein the alert is issued if the quantity of physical waking is below a walking threshold.

7. A method as in claim 1, wherein the monitor device is a wearable device.

8. A method as in claim 7, wherein the monitor device is a pedometer.

9. A method as in claim 1, wherein the quality of life data is manually provided by the post-operative patient.

10. A method as in claim 1, wherein the health parameter data is received from the monitor device via a mobile application.

11. A method as in claim 1, further comprising tabulating trends in the quality of life data and the health parameter data.

12. A method as in claim 1, further comprising verifying that the post-operative patient recently underwent a surgical procedure.

13. A method as in claim 1, further comprising assessing at least one of the quality of life data and the health parameter data pursuant to a MD Anderson Symptom Inventory (MDASI).

14. A mobile application for monitoring a post-operative patient, the mobile application including software for performing at least the following steps:
receiving health parameter data from a monitor device attached to the post-operative patient, the health parameter data being automatically generated by the monitor device and being indicative of a physical parameter of the post-operative patient;
receiving quality of life data from the post-operative patient, the quality of life data being generated by the post-operative patient;
sending the health parameter data and the quality of life data to a data center for analysis related to post-operative care of the post-operative patient.

15. A mobile application as in claim 14, wherein the health parameter data is automatically generated by a monitor device attached to the post-operative patient.

16. A system for managing post-operative care of a patient, comprising:
a wearable device that can be worn by a patient, the wearable device configured to sense or monitor health parameter data of the patient;
a mobile application communicatively coupled to the wearable device, the mobile application configured to obtain the health parameter data from the wearable device and to also obtain quality of life data from the patient;
a data center communicatively coupled to the mobile application, wherein the data center receives the health parameter data and the quality of life data from the mobile device for analysis related to post-operative care of the patient.

17. A system as in claim 16, wherein the wearable device is a pedometer.

18. A system as in claim 16, wherein the health parameter data comprises at least one of a heart rate of the post-operative patient, skin or ambient temperature of the post-operative patient, body position of the post-operative patient, ECG of the post-operative patient, physical activity of the post-operative patient, daily walking steps of the post-operative patient, pulse oximetry of the post-operative patient, blood pressure of the post-operative patient, sleep activity of the post-operative patient, and blood glucose of the post-operative patient.

19. A system as in claim 16, wherein the quality of life data comprises at least one of data related to mobility of the post-operative patient, self-care of the post-operative patient, usual activities of the post-operative patient, pain and discomfort of the post-operative patient, and anxiety or depression of the post-operative patient.

20. A system as in claim 16, wherein the mobile application wirelessly communicates with the data center.
